(19) United States
(12) Patent Application Publication
(10) Pub. No.: US 2020/0405655 A1
(43) Pub. Date: Dec. 31, 2020

(54) METHOD FOR ACCELERATING WOUND HEALING

(71) Applicant: Tanya Anderson, Lakeville, MN (US)

(72) Inventor: Tanya Anderson, Lakeville, MN (US)

(21) Appl. No.: 16/976,030

(22) PCT Filed: Feb. 26, 2019
(86) PCT No.: PCT/US2019/019579

§ 371 Date: Aug. 26, 2020

Related U.S. Application Data
(60) Provisional application No. 62/635,758, filed on Feb. 27, 2018.

(51) Int. Cl. A61K 9/70; A61P 17/02

(52) U.S. Cl. CPC A61K 9/7076; A61P 17/02; A61K 9/7069

(57) ABSTRACT

Disclosed is a method and composition for accelerating wound healing comprising applying a dressing directly on a subject's wound immediately or no later than 72 hours following injury. The dressing reduces or minimizes scarring, prevents or reduces infection, inhibits or reduces internal bleeding, inhibits or reduces external discharge, provides protection from sun and/or UV rays or a combination thereof, thereby accelerating wound healing.

### CROSS REFERENCE TO RELATED APPLICATIONS

This application claims the benefit of U.S. Provisional Patent Application Ser. No. 62/635,758, filed Feb. 27, 2018, the disclosure of which is incorporated in its entirety herein by reference.

### BACKGROUND

Wound healing is a complex process involving cells, the extracellular matrix (ECM) components, and the cellular microenvironment. Most wound healing involves the repair or replacement of damaged tissues. The precise nature of the repair or replacement depends upon the tissues involved; although most wound healing processes involve certain phases or events: hemostasis, inflammation, proliferation, and remodeling. Wound healing begins as soon as the tissue is injured. As blood spills into the site of injury, the platelets come into contact with exposed collagen and extracellular matrix, releasing clotting factors, to achieve hemostasis as well as essential growth factors (GFs) and cytokines to repair the wound. These GFs include platelet-derived GF, transforming GF beta, vascular endothelial GF (VEGF), platelet-derived epidermal GF, insulin-like GF-I and basic fibroblast GF (bFGF) to name a few. Neutrophils followed by the macrophages then begin to phagocytose bacteria, damaged tissue and other foreign materials, as part of the inflammatory phase. Following this, fibroblasts migrate and begin the proliferative phase which deposits new extracellular matrix and collagen. During the final remodeling phase, the newly laid collagen matrix becomes organized and cross-linked. Numerous cell-signaling events and cytokines are required to orchestrate these events,

There is a continuing need for compositions and methods which accelerate wound healing.

### SUMMARY

Described is a method and composition for accelerating wound healing. In one embodiment is a method for accelerating wound healing comprising applying a dressing directly on a subject's wound immediately or no later than 72 hours following injury.

The dressing reduces or minimizes scarring, prevents or reduces infection, inhibits or reduces internal bleeding, inhibits or reduces external discharge, provides protection from sun and/or UV rays or a combination thereof, thereby accelerating wound healing.

### BRIEF DESCRIPTION OF THE DRAWINGS

FIG. 1A shows the reduction of scarring of an incision when an embodiment of the dressing is applied or not applied.

FIG. 1B is a close-up comparing the scar reduction when an embodiment of the dressing is applied or not applied in the area.

FIG. 1C is a close-up showing scar reduction when an embodiment of the dressing is applied.

FIG. 2 is a close-up comparing the scar reduction when an embodiment of the dressing is applied or not applied in the area.

FIG. 3A-3C shows discharge on gauze compared to discharge using an embodiment of the dressing.

FIG. 4A-B shows discharge using an embodiment of the dressing.

FIG. 5 shows a difference in the rate of healing and internal bleeding under an embodiment of the dressing that was applied 20 hours post operation.

FIG. 6A-B shows evidence of an embodiment of the dressing's ability to block sun and/or UV rays.

### DETAILED DESCRIPTION

Although the present disclosure provides references to preferred embodiments, persons skilled in the art will recognize that changes may be made in form and detail without departing from the spirit and scope of the invention. Reference to various embodiments does not limit the scope of the claims attached hereto. Additionally, any examples set forth in this specification are not intended to be limiting and merely set forth some of the many possible embodiments for the appended claims. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

As used herein the term “dressing” refers to any material applied to the wound that provides protection, support, hydration, oxygen transmission, a reduction in inflammation, sun and/or UV protection, infection prevention, inhibition or reduction of internal bleeding, inhibition or reduction of external discharge, accelerated scar maturity or combinations thereof.

As used herein the term “accelerating” when used in context of wound healing refers causing the speeding up or moving faster the development or progress of wound healing compared to a wound not applied with a dressing.

As used herein the term “subject” refers to any mammal including, but not limited to humans.

As used herein the term “scar” when used in context of the dressing refers to hypertrophic scars or keloids or any area of fibrous tissue that replaces normal skin after an injury. or a combination thereof.

As used herein the term “wound” refers to injuries to tissue including the cutaneous and subcutaneous tissue, initiated in any manner, for example by surgery.

Scar formation in response to cutaneous injury is part of the natural wound healing process. Wound healing is a lengthy and continuous process, although it is typically recognized as occurring in stages. The process begins immediately after injury, with the hemostasis stage and progresses to the inflammatory stage. During the inflammatory stage, which typically lasts from two days to one week (depending on the wound) damaged tissues and foreign matter are removed from the wound. The proliferative stage occurs at a time after the inflammatory stage and is characterized by fibroblast proliferation and collagen and proteoglycan production. It is during the proliferative stage that the extracellular matrix is synthesized in order to provide structural integrity to the wound. The proliferative stage usually lasts about four days to several weeks, depending on the nature of the wound, and it is during this stage when hypertrophic scars usually form. The last stage is called the remodeling or maturation stage. During the remodeling stage the previously constructed and randomly organized matrix is remodeled into an organized structure that is highly cross-linked and aligned to increase mechanical strength. Accelerating the rate at which a wound moves through the inflammatory and proliferative stage and reaches the maturation stage is a significant component to wound healing thereby minimizing scar formation.

In the wound healing process, typically a normal scar results. If pathologic increase in scar formation results, it forms a hypertrophic scar or a keloid or any area of fibrous tissue that replaces normal skin after an injury.

While the histological features characterizing hypertrophic scars have been well documented, the underlying pathophysiology is not well known. Hypertrophic scars are a side effect of excessive wound healing, and generally result in the overproduction of cells, collagen, and proteoglycans. Thus, accelerated wound healing is desirable in the effort to minimize scar formation. Typically, these scars are raised and are characterized by the random distribution of tissue bundles. The appearance (e.g., size, shape, and color) of these scars varies depending on the part of the body in which they form, and the underlying ethnicity of the person affected. Hypertrophic scars are common and may occur following any injury to the cutaneous or subcutaneous tissue. It has been shown that mechanical stress may increase hypertrophic scarring (See U.S. Patent Application Publication 2006/0037091 (U.S. patent application Ser. No. 11/135,992 entitled “Method for Producing Hypertrophic Scarring Animal Model for Identification of Agents for Prevention and Treatment of Human Hypertrophic Scarring,” filed May 24, 2005).

Keloids are typically characterized as tumors consisting of highly hyperplastic masses that occur in the dermis and adjacent subcutaneous tissue in susceptible individuals, most commonly following trauma. Keloids are often more severe than hypertrophic scars, since they tend to invade normal adjacent tissue, while hypertrophic scars tend to remain confined within the original scar border.

Previous attempts to treat scars and keloids have included surgery, silicone dressings, steroids, x-ray irradiation, and cryotherapy. Each of these techniques has disadvantages. Perhaps the biggest disadvantage is that none of them effectively prevent or ameliorate the formation of scars or keloids in the first instance. That is, these techniques have primarily been used to treat scars after they are already well established. Provided herein, in some embodiments are methods of minimizing the initial formation of excessive scar formation. In some embodiments, the methods promote wound healing.

Providing support to the wound and accelerating the rate at which a wound reaches the maturation wound healing phase are significant components to scar control. Hydration is another significant component to scar control. After the initial injury there is a robust inflammatory cascade that results during which much of the downstream outcome of scars develops. There is tissue loss in the wound area during the inflammatory phase which leaves an area of tissue which is devoid of matrix that is subsequently replaced with scar tissue in the remodeling phase. The amount of inflammation directly correlates with the amount of tissue loss and subsequently the amount of scar tissue that forms. Reducing the time spent in the inflammatory phase and/or the severity of inflammation is thus a goal to minimize scar formation thereby promoting wound healing. Tension on a wound from many directions or intermittently promotes inflammation and often results in a hypertrophic scar, initiated possibly by lymphocytes or a prolonged inflammatory process. To counteract this tension, supporting the wound is desirable.

As the majority of inflammation occurs in the first 72 hours after a wound occurs, it is desirable that a dressing be applied immediately or substantially immediately after the wound occurs. The sooner the dressing is applied to the wound, the faster the dressing may provide support to the wound, reducing tension (mechanical stress) on the wound, thereby reducing inflammation and accelerating wound maturation. The practice in the industry is typically to wait to apply a dressing for a period of time after the injury occurs, for example the advised application date might be after the sutures are removed or the injury is no longer draining or some other later suggested application date. Although later than an immediate application can still have a desirable outcome, the amount of time a wound is exposed to external infectors the greater the likelihood of acquiring an infection. Infection will slow down the healing process and may result in any number of undesirable outcomes that may include, but are not limited to, increased or prolonged inflammation, prominent scars and mortality. Thus, avoiding infection is a primary goal in the medical field. In addition, evidence of the rate at which a wound is healing can at times be found in the reduction of internal bleeding and the minimization of external discharge. Thus, the earliest possible time when reduction in internal bleeding and external discharge can be achieved is desirable. Likewise, the amount of inflammation that is allowed to occur correlates with the final scar outcome. In other words, immediate application of the dressing has a greater impact on accelerating wound healing, preventing or reducing infection, and minimizing scar formation than later application of the dressing. Therefore, in some embodiments, the dressing is applied during the inflammatory stage of the wound healing process. In some embodiments, the method includes applying a dressing on a wound immediately or no later than 72 hours following injury. In some embodiments, the dressing is applied within one hour to within 72 hours of injury, from 10 hours to within 24 hours, 16 hours to within 48 hours, 36 hours to within 52 hours or 48 hours to 72 hours. In some embodiments, the dressing is applied within 2 hours, 4 hours, 8 hours, 16 hours or within 24 hours of injury.

In some embodiments, the dressing is applied to a wound where the wound is an open wound. In some embodiments, the dressing is applied to a wound where the wound is partially or incompletely closed or initially been closed (e.g., by suturing following surgery). The dressing is applied directly over the wound. In some embodiments, the dressing is applied directly over an incision that has been sutured or otherwise closed after surgery.

In some embodiments, the dressing is configured to shield a wound from exposure to various stresses such as infection and UV exposure. In some embodiments, the dressing may be made of UV blocking materials. In some embodiments, the dressing contains UV blocking agents (e.g., titanium dioxide (TiO2), kaolin, zinc oxide (ZnO), calcium carbonate, and magnesium oxide). In other embodiments UV protecting agents include bemotrizinol, avobenzone, bisoctizole, benzophenone-3 (BZ-3, oxybenzone), and octocrylene, ecamsule (terephthalylidene dicamphor sulphonic acid), dometrizole trisiloxane, bemotrizinol, and bisoctrizole.

In some embodiments, the dressing is a barrier to (or inhibits the exposure to) externally contracted infections (e.g. healthcare acquired). Skin is a natural barrier to defend the body against external organisms that can cause infection. When the skin is broken (when a wound or injury occurs) the risk of infection (and thus additional complications that may include but are not limited to increased or prolonged inflammation, prominent scarring and mortality) is greatly increased. Providing a dressing as a barrier to these external organisms is essential to ensuring accelerated healing is achieved and the risk of infection and the level of inflammation are kept to a minimum.

In some embodiments, a dressing is applied to eliminate or reduce the risk (or occurrence) of healthcare acquired infections, also known as nosocomial infections.

In some embodiments, a dressing is applied to prevent tissue movement and tension.

In some embodiments, the dressing reduces or eliminates the amount of internal bleeding thereby accelerating the wound healing process. Reducing the amount of internal bleeding will reduce the amount of inflammation at the wound site and thus have an impact on the resulting scar formation. Preventing internal bleeding is desirable to accelerate wound healing and minimize scar formation.

Hydration is another significant component to accelerate the wound healing process and to minimize scar formation. Dry wounds heal slower and thus tend to scar more. Therefore, normalizing transepidermal water loss (TEWL) and moisture vapor transmission rate (MVTR) is important to scar control. In some embodiments, the dressing promotes an optimal TEWL and MVTR environment for healing. In other embodiments, the dressing encourages moisture vapor permeation and transmission. In other embodiments, the dressing provides an effective barrier to pathogens, while permitting moisture that accumulates to evaporate.

Oxygen has a significant effect on the wound healing process. For example, when an area of the body does not receive an adequate amount of oxygen, a condition known as hypoxia, it can slow and even halt the healing process. Adequate wound tissue oxygenation is required for optimal wound healing and thus scar reduction. In some embodiments, the dressing promotes adequate tissue oxygenation.

The dressing once applied to the wound of a subject immediately or substantially immediately after injury may then be removed after a suitable time period. For example, the time period of application to the wound may be determined by the wound type. In other embodiments, the time period is determined by following the wound healing process. In other embodiments, the time period is determined by when the dressing spontaneously separates from the wound (spontaneous separation). In other embodiments, the time period is determined by the anticipation of the spontaneous separation of the dressing. In other embodiments, the time period is determined by the need for medical care of the wound. In some embodiments, the dressing is applied for 4 weeks to 24 weeks, from 4 weeks to 20 weeks, from 8 weeks to 24 weeks, from 12 weeks to 20 weeks, from 12 weeks to 24 weeks, from 16 weeks to 24 weeks, or 20 weeks to 24 weeks. The time reported herein reflects the time the wound site is covered by a dressing. For example, when a dressing is applied for 24 weeks, it means that the dressing once applied on the wound is present on the wound for the entire time period. In other words the dressing once applied for 24 weeks is on the wound 24 hours a day, 7 days a week for the entire 24 weeks. The dressing may be removed and a fresh, second dressing may be immediately applied or followed by a third dressing or any suitable number of dressings as needed for total time period of 24 weeks. In other words, any number of dressings may be applied during the requisite time period so long as when a dressing is removed, it is immediately replaced by another dressing for the total number of days required. The user is able and can conduct daily activities (e.g., bathing, swimming, sleeping) while the dressing is in place.

In some embodiments, heat is applied to the dressing after application to ensure the dressing is secure. In some embodiments, this heat is simply from the subject's hand. In some embodiments, this heat can be applied for 10-60 seconds or 20-40 seconds.

The dressing and methods described herein can be adapted for a variety of wound sizes, wound thickness, and for different skin thickness, e.g., the dressing may be configured for use in different areas of the body. In addition, the dressing and methods described here can be adapted to accelerate wound healing and minimize scar formation in any type of skin, body location, age, race, or condition.

In some embodiments, the dressing may be removably secured to skin surface. Similarly, the dressing may be any shape or size suitable to cover the wound site. In some embodiments, the dressing is applied in the direction of the wound and overlaps each side of the wound by a sufficient margin to ensure the wound edges are securely held in place.

In some embodiments, the dressing when applied can provide slight pressure around the wound improving the healing and minimizing scar formation. In some embodiments, the dressing is used to accelerate wound healing and reduce or minimize the formation of scars (e.g. hypertrophic scars and keloids).

In some embodiments, the dressing is a sticky, pliant, skin friendly adhesive composition. The dressing is suitably soft, flexible and non-irritating to the skin. In some embodiments, the dressing includes a flexible, backing layer along one of its faces. Along the opposite side of the backing layer is a body-contacting face. The body-contacting face of the dressing includes an adhesive. The adhesive of the body-contacting face may be used to secure the body contacting face with the other face of the backing layer. In some embodiments, the two faces of the dressing are secured to one another by interposing a porous or microporous medical-grade adhesive, such as a pressure-sensitive acrylic adhesive, between the two. In such a case, the adhesive should be applied or formulated in any of a variety of known ways so that it does not block the transmission or permeation of moisture and oxygen through the backing layer.

In some embodiments, the backing layer is made of polyethylene woven fibers. In some embodiments, the backing layer is thermoplastic. In other embodiments, the backing layer is biocompatible silicone polymers, shape memory polymers and the like. In some embodiments, the backing layer is composed of thermoplastic elastomers such as polyurethane films, copolyester films, nylons, vinyl, acetate taffeta, or any other suitable pliable material. Commercially available elastomers include the type marketed under the designation Hytrel by DuPont (Wilmington, Del.) or polyether-block amide film marketed under the designation Pebax by Elf Atochem (Philadelphia, Pa.). In other embodiments, non-elastomeric films and non-woven materials are used.

In some embodiments, the backing layer is porous. In other embodiments, the backing layer is microporous, soft, flexible, and heat-sealable. It may, for example, take the form of a textured thermoplastic film with a multiplicity of tiny perforations that allow for the transmission of gases, including water vapor. In some embodiments, the backing layer is breathable (e.g., gas-transmissible) nonwoven fabric composed of spunbonded or meltblown thermoplastic fibers. One such product is a spunbonded low density polyethylene nonwoven fabric available under the designation “Daltex” 6080-A1-UPE from Don & Low Ltd., Forfar, Scotland, but other soft, porous, heat-sealable nonwoven fabrics are available and may also be used. In some embodiments, flexible and resilient thermoplastic foam materials of open or semi-open cell structure are suitable to fabricate the backing layer.

In still other embodiments, suitable gas-permeable, microporous materials may be formed from a single layer of a nonwoven material or a multilayer material including a polymeric film and/or nonwoven material.

In some embodiments, the backing layer is a thin elastomeric film having moisture vapor transmission characteristics generally approximating those of healthy skin. In some embodiments polyurethane film is in the thickness range of about 1 to 2 millimeters. Other polymeric films such as copolyesters or copolyamides may be used if they have similar permeability characteristics. In some embodiments, the backing layer is in the form of microporous fabric, such as microporous woven fibers. Vapor-permeable films are suitable because their permeability is by diffusion, thereby assuring that the dressing itself is an effective barrier to liquids and pathogens, including bacteria and viruses, despite the fact that moisture accumulated by the body-contacting layer is allowed to escape by evaporation and diffusion through the permeable backing layer.

In some embodiments the adhesive is a medical grade, non-woven acrylic adhesive that is pressure sensitive.

In some embodiments the adhesive is a medical grade, pressure sensitive adhesive such as polyisobutene, natural rubber adhesive, acrylate and methacrylate adhesive or silicone adhesive. In some embodiments, the adhesive includes elastomers. For example, the elastomers include polyisobutylenes with which additives such as butyl rubber may be blended. In some embodiments, the elastomer may contain a styrene block copolymer component to help provide extensibility and recoverability from modular strains. In addition, such dressings may include mineral oil, to increase stretchability and adhesiveness of the blend, and suitable tackifying agents and antioxidants.

In some embodiments, the dressing includes hydrogels (crosslinked polymers having about at least 50% water). In some embodiments, some hydrogels with adhesive properties such as adherent hydrogels are used.

In some embodiments, the adhesive contains substantially homogeneous mixture of selected components forming an adhesive viscoelastic continuous phase in which swellable hydrocolloid particles are dispersed. In some embodiments, the continuous phase is a blend of elastomers. In some embodiments, the elastomers include entirely or substantially entirely about 2 to 15 percent or 3 to 7 percent by weight of one or more high molecular weight polyisobutylenes and about 5 to 20 percent by weight or 7 to 14 percent of one or more styrene block copolymers. “High molecular weight” refers to a polyisobutylene having a viscosity average molecular weight within the range of about 750,000 to 2,350,000 or about 1,000,000 to 1,900,000 as determined from intrinsic viscosity measurement in diisobutylene at 20° C. Such polyisobutylenes are commercially available and are known, for example, under the designations Vistanex MM-L80, MM-L100, MM-L120, and MM-L140 from Exxon Corp., Houston, Tex.

In some embodiments, a styrene block copolymer or copolymers are suitable for blending with such high molecular weight polyisobutylene(s). In some embodiments, the styrene block copolymer includes styrene-olefin-styrene block copolymers, styrene-isoprene-styrene or styrene-butadiene-styrene block copolymers, or combinations thereof. In some embodiments, commercial styrene block copolymers are available from Shell Chemical and other suppliers. A styrene-isoprene-styrene block copolymer marketed as Kraton 1107 (Shell Chemical) are suitable, as are other Kraton copolymers, such as Kraton 1100 series from Shell Chemical Co such as, 1101, 1102 may be used. In yet other embodiments, elastomer includes a styrene-isoprene-styrene block copolymer (e.g., “Cariflex” Tr-1107, from Shell Chemical Co.) and other ABA block copolymers, such as ethylene-propylene block copolymers known as EPR rubbers have also been included in adhesive compositions for increasing the elastomeric properties of the dressing materials.

In some embodiments, other adhesives that are capable of transmitting moisture vapor therethrough are used such as acrylic adhesives. Such transmission may result from the permeability of the acrylic adhesive layer because of micropores formed therein or as a result of the capability of the adhesive layer to allow the diffusion of gas therethrough. In some embodiments, the adhesive is a medical grade pressure sensitive adhesive. In other embodiments, the adhesive is a vinyl-acrylic emulsion.

In some embodiments, the adhesive includes plasticizers. In some embodiments, the plasticizer includes petrolatum or mineral oil. Petrolatum is relatively viscous and non-flowing at room temperature, as compared to mineral oil, and these properties are desirable in achieving a pliant viscoelastic, and cohesive dressing composition. In some embodiments, the dressing composition contains about 6 to 20 percent by weight of petrolatum or mineral oil plasticizer, or about 8 to 15 percent by weight.

In some embodiments, the dressing also contains one or more hydrocarbon tackifier resins homogeneously distributed in and forming part of the continuous phase of the composition. In some embodiments, an aliphatic hydrocarbon resin tackifier commercially available from Hercules Inc. (Wilmington, Del.) as Piccotac 95 is used. In other embodiments, other tackifiers such as the trimethylol propane esters of rosin (Staybelite Ester 10 from Hercules) or the pentaerythritol esters of rosin (Pentalyn H from Hercules) are used. In some embodiments, still other tackifiers suitable for use in the dressing are beta pinene or cyclopentadiene resins that are also commercially available. In some embodiments, the tackifier content falls within the range of about 10 to 35 percent by weight, or about 20 to 30 percent by weight.

In some embodiments, the dressing includes an adhesive formulated for adhering to moist tissue. Such an adhesive includes a silicone elastomer, a hydrophilic component, a superabsorbent polymer or a combination thereof. In some embodiments, the adhesive is formulated with an addition-curing two component silicone elastomer, a crosslinked polyacrylic acid polymer, and a sodium polyacrylate based superabsorbent polymer. Dressings using such an adhesive can adhere to and seal around the wound, and move with the wound during use.

In some embodiments the discontinuous phase includes swellable materials. Such swellable materials include one or more water soluble hydrocolloid gums which are capable of absorbing moisture and preventing such moisture from disrupting adhesion to skin surfaces. Hydrocolloids include gums commonly used such as sodium carboxymethylcellulose, pectin, gelatin, guar gum, locust bean gum, gum karaya, and mixtures thereof. In some embodiments, the hydrocolloid content ranges from about 35 to 65 percent or from about 40 to 55 percent by weight. In some embodiments, the hydrocolloid content includes pectin and sodium carboxymethylcellulose in a ratio of approximately 2 to 1.

In other embodiments, the adhesive can be a pressure sensitive adhesive such as silicone based pressure sensitive adhesives.

In other embodiments, the adhesive compositions are skin friendly, possess good adhesive strength and good cohesive strength, and include gel-like properties, e.g. super absorbents and/or liquid swellable gel forming materials.

In other embodiments, the pressure-sensitive adhesive composition comprises a triblock copolymer and a superabsorbent material and self-adhering wound dressings comprising the same.

In an embodiment, an adhesive composition for moist tissue may be formulated with about 70 weight percent (wt. %) to about 99 wt. % of silicone adhesive and about 1 wt. % to about 30 wt. % of hydrophilic components, about 80 wt. % to about 98 wt. % of silicone adhesive or about 2 wt. % to about 20 wt. % of hydrophilic components, about 85 wt. % to about 97 wt. % of silicone adhesive and about 3 wt. % to about 15 wt. % of hydrophilic components. In some embodiments, the adhesive composition may also include about 0.05 wt. % to about 5 wt. % of fibers, 0.1 wt. % to about 2 wt. % of fibers, 0.5 wt. % to about 1 wt. % of fibers. Suitable fibers include fibrillated high density polyethylene (HDPE) fibers. The adhesive composition may also include ceramide. In some embodiments the ceramide is about 0.05 wt. % to about 1 wt. % of ceramide, about 0.1 wt. % to about 0.5 wt. % of ceramide.

Suitable silicone adhesives for adhesive compositions for moist tissue include two-part addition curing silicone compositions that cure at room temperature, which may also be referred to as RTV-2 silicone. An example of suitable RTV-2 silicones is a two-part platinum (Pt) catalyzed silicone gel elastomer composition including component A comprising Pt and unsaturated polymers with a vinyl group, such as R—CH═CH2, and component B having silicone reactive groups, such as R′—SiH, which participates in Pt catalyzed addition reaction, known as hydrosilylation.

In an embodiment, an adhesive composition for moist tissue may include a two-part Pt catalyzed silicone elastomer composition including component A comprising Pt and vinyl functional polymers (R—CH═CH2) and component B comprising silicone hydride groups (—SiH), and hydrophilic components. The adhesive composition may be formulated with a sufficient quantity of the two-part Pt catalyzed silicone elastomer to hold the shape and structure after the adhesive composition is molded and cured. Further, the adhesive composition may be formulated with sufficient quantities of hydrophilic components.

In some embodiments, an adhesive composition for moist tissue may comprise about 85 wt. % to about 97 wt. % of a two-part Pt catalyzed silicone elastomer and about 3 wt. % to about 15 wt. % of hydrophilic components, wherein the hydrophilic components may comprise about 1 wt. % to about 14 wt. % of a crosslinked polyacrylic acid polymer and about 3 wt. % to about 14 wt. % of a crosslinked polyacrylic acid polymer.

The dressing can include additional materials that aid in the wound healing process. For example the additional materials may include growth factors, vitamins (e.g. vitamin E or combinations thereof), antioxidants, enzymes such as elastase to degrade the extra cellular matrix, proteases such as aspartate, serine, and metalloproteases that are capable of digesting and remodeling tissue, inhibitors of enzymes such as tissue inhibitors of metalloproteases, antibiotics, antifungals, and combinations thereof. In some embodiments, delivery of the additional materials can be controlled by time-release, e.g., by encapsulating or embedding the additional materials in a time-release formulation, such as a drug delivery polymer or depot. Agents that are known to increase cell growth or vascularization or reduce inflammation may also be included such as platelet derived growth factor, epidermal growth factor, anti-inflammatory drugs, vascular endothelial growth factor (VEGF), fibroblast growth factor or combinations thereof. In some embodiments, the dressing includes ceramide.

Suitable commercially available dressings that may be used in the method for reducing or minimizing scarring include SOFTFLEX™, FLEXTEND M™ FORMAFLEX™, FLEXTEND™, FLEXWEAR™, and CERAPLUS™, all available from Hollister Incorporated, USA. In some embodiments, the dressing is commercially available from Hollister, New Image Convex FLEXTEND™ Skin Barrier cut-to-fit (Hollister product #14803, extended wear).

Some additional non-limiting embodiments are provided below to further exemplify the present disclosure:

A method for reducing or minimizing scarring, comprising applying a dressing directly on a subject's wound immediately or no later than 72 hours following injury.

The method of embodiment 1, wherein the applying the dressing is within 1 hour of the injury.

The method of embodiment 1, wherein the applying the dressing is within 2 hours, 4 hours, 8 hours, 16 hours, or 24 hours of injury.

The method as in any one of embodiments 1-3, wherein the wound is an incision.

The method as in any one of embodiments 1-3, wherein the wound is an incision followed by suturing after surgery.

The method as in any one of embodiments 1-5, wherein the applying the dressing is for about 4 weeks to about 24 weeks.

The method as in any one of embodiments 1-5, wherein the applying the dressing is for about 16 weeks to about 24 weeks.

The method as in any one of embodiments 1-7, wherein the applying the dressing to the wound prevents tissue movement and decreases tension.

The method as in any one of embodiments 1-8, wherein the dressing allows moisture vapor permeation.

The method as in any one of embodiments 1-9, wherein the dressing allows oxygen permeation.

The method as in any one of embodiments 1-10, wherein the dressing contains UV blocking components.

The method as in any one of embodiments 1-11, wherein the dressing is a barrier to externally contracted infections.

The method as in any one of embodiments 1-12, wherein the dressing comprises a backing film and a layer coating the backing film, wherein the backing film comprises polyethylene woven fibers.

The method as in any one of embodiments 1-13, wherein the layer coating the backing film comprises a non-skin irritating adhesive.

The method as in any one of embodiments 1-14, wherein the adhesive comprises a pressure sensitive, medical grade adhesive.

16. The method as in any one of embodiments 1-15, wherein the adhesive comprises a non-woven acrylic adhesive.

17. The method as in any one of embodiments 1-15, wherein the adhesive comprises a vinyl-acrylic polymer emulsion.

18. The method as in any one of embodiments 1-17, wherein the dressing comprises antioxidants, ceramides, growth factors, vitamins, enzymes, antibiotics, antifungals, and a combination thereof.

19. The method as in any one of embodiments 1-18, wherein the subject is human.

20. The method as in any one of embodiments 1-19, wherein the dressing comprises the materials contained in the Hollister, New Image Convex FLEXTEND™ (extended wear) Skin Barrier or any other Hollister Skin Barrier.

### EXAMPLES

The following examples are intended to illustrate different aspects and embodiments of the invention and are not to be considered limiting the scope of the invention. It will be recognized that various modifications and changes may be made without following the experimental embodiments described herein, and without departing from the scope of the claims.

### Example 1

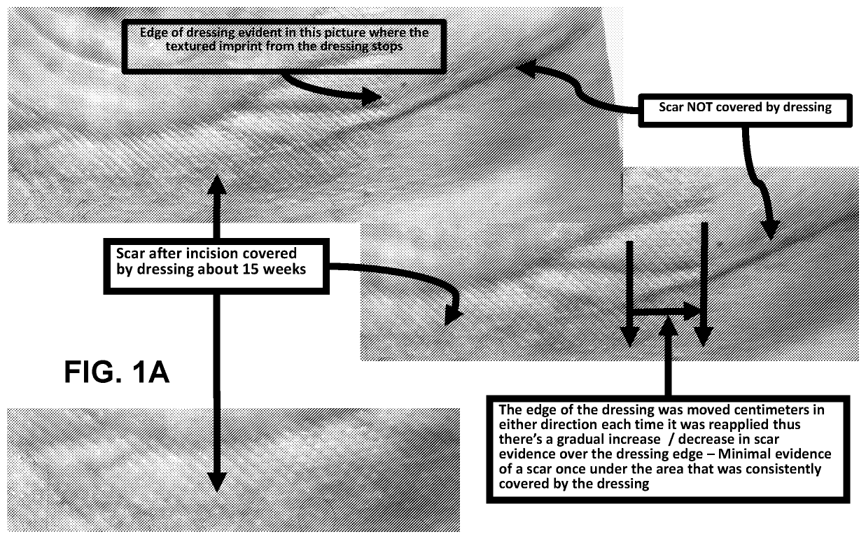

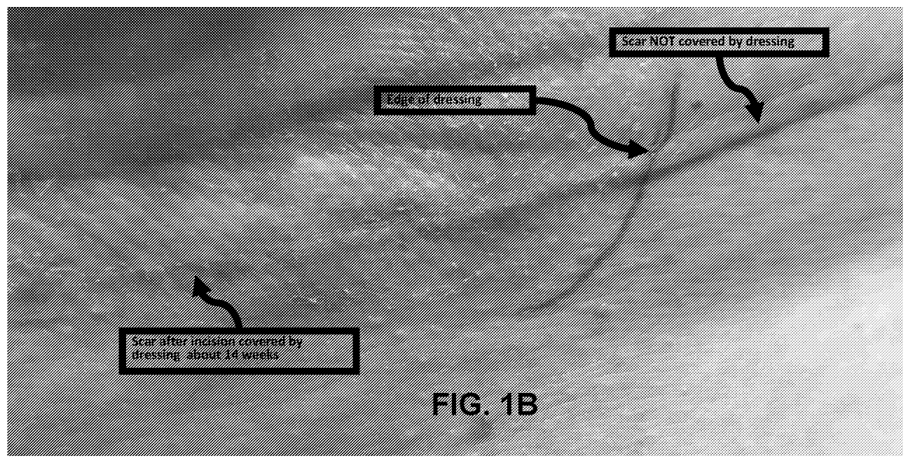

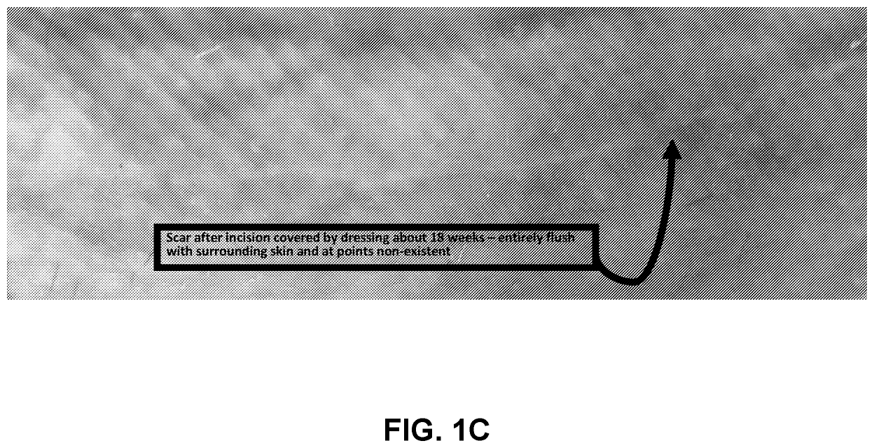

A dressing obtained from Hollister, New Image Convex FLEXTEND™ Skin Barrier cut-to-fit (Hollister product #14803, extended wear) was cut to the appropriate size and placed on parts of an incision immediately after surgery. The dressing was removed and a fresh, new dressing was applied as needed. FIGS. 1A and 1B show the reduction in scaring from 14 to 15 weeks post operation in areas covered by the dressing compared to the areas in which the incision was not covered by the dressing. FIG. 1C shows the reduction in scaring at 18 weeks after surgery in an area covered by the dressing.

### Example 2

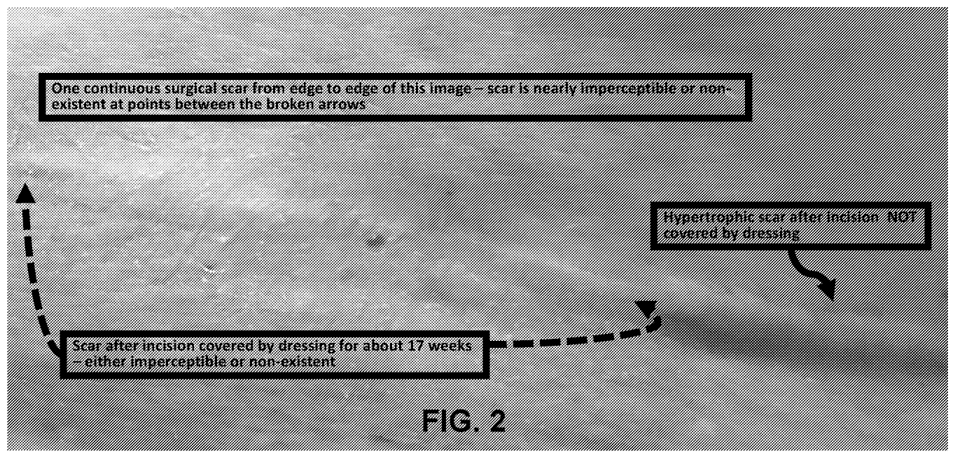

A dressing obtained from Hollister, New Image Convex FLEXTEND™ Skin Barrier cut-to-fit (Hollister product #14803, extended wear) was cut to the appropriate size and placed on parts of an incision 20 hours after surgery. The incision was covered by the dressing for a total period of about four months. The dressing was removed and a fresh, new dressing was applied as needed. FIG. 2 shows the reduction in scaring after about 17 weeks in an area covered by the dressing.

### Example 3

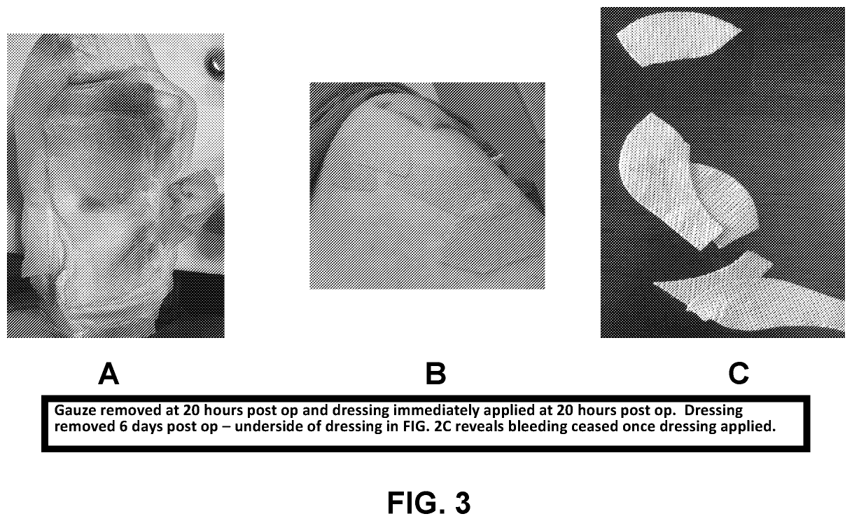

FIG. 3A shows the discharge on gauze from an incision immediately applied after surgery and removed 20 hours post operation. FIG. 3B shows application of a dressing obtained from Hollister, New Image Convex FLEXTEND™ Skin Barrier cut-to-fit (Hollister product #14803, extended wear), which was cut to the appropriate size and placed on an incision 20 hours post-surgery. The incision was covered by the dressing for a total period of 6 days. As can be seen in FIG. 3C, by the absence of blood on the underside of the dressing, the bleeding stopped once the dressing was applied.

### Example 4

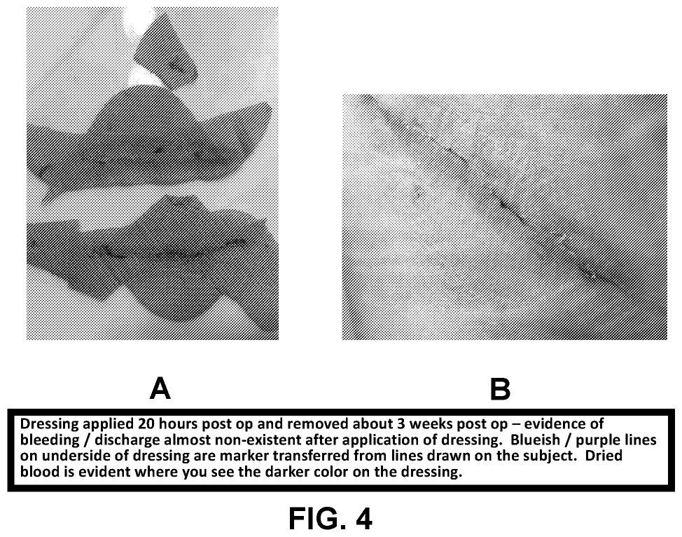

A skin barrier obtained from Hollister, New Image Convex FLEXTEND™ (extended wear) Skin Barrier, was cut to the appropriate size and placed on parts of an incision 20 hours post-surgery. The incision was covered by the dressing for a total period of 19 days. FIG. 4A shows the underside of the dressing when removed at 19 days. Almost no bleeding is evident on the underside of the dressing. The purple/blueish lines in FIG. 4A is marker that transferred off of the subject's body onto the dressing. The dried blood is seen only in the darker part of the underside of the dressing. FIG. 4B shows the corresponding lack of discharge/blood residue on the incision at the time the dressing was removed and as shown in FIG. 4A.

### Example 5

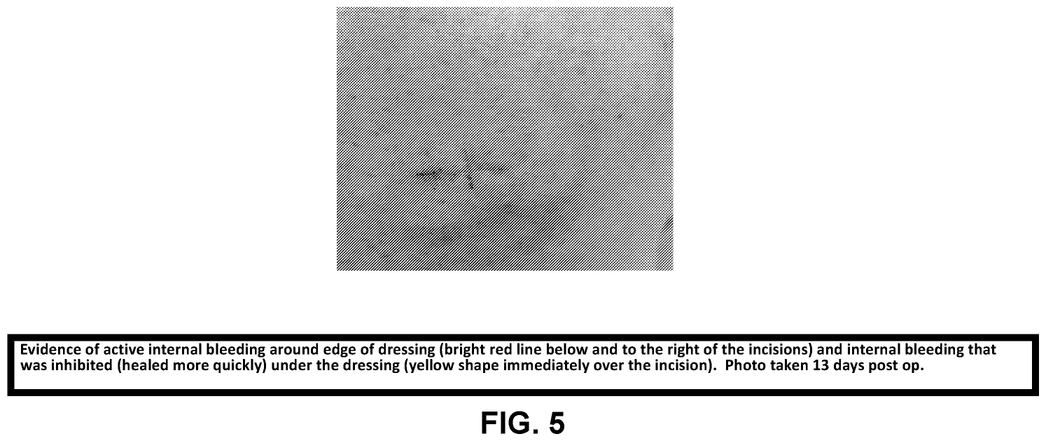

A dressing obtained from Hollister, New Image Convex FLEXTEND™ (extended wear) Skin Barrier, was cut to the appropriate size and placed on an incision 20 hours post-surgery. FIG. 5 shows a difference in the rate of healing and the effect on internal bleeding under a dressing that was removed 12 days after surgery. FIG. 5 shows the discoloration around the edges of the dressing that was applied 20 hours post-surgery. The red line around the wound (to the right and below the wound) exactly follows the edge of the dressing. Anywhere that appears red was not covered by the dressing. The red discoloration indicates internal bleeding around the edges of the dressing. Lack of discoloration (bruising under the dressing) indicates internal bleeding stopped more quickly and healing occurred more quickly under the dressing.

### Example 6

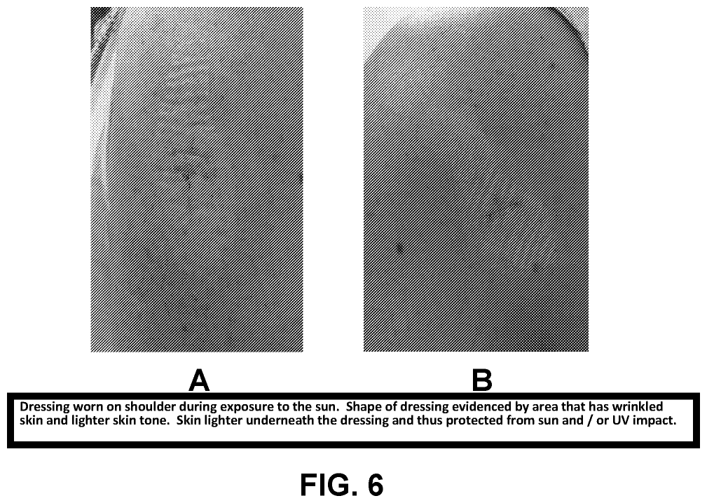

FIG. 6A-B shows evidence of the dressing's (New Image Convex FLEXTEND™ Skin Barrier cut-to-fit Hollister product #14803) ability to block the sun and UV rays.

Subject had the dressing in place while in the sun during the days preceding the removal of the dressing. Skin is more wrinkly under the dressing due to ridges on the underside of the dressing thus irregular dressing edges evident where the more wrinkly skin stops. Darker skin tone, due to exposure to the sun, is evident around the edges of the dressing. Lighter skin tone under the dressing indicates the dressing has UV protection properties.

1. A method for accelerating wound healing comprising applying a dressing directly on a subject's wound immediately or no later than 72 hours following injury.

2. The method of claim 1, wherein the accelerating wound healing comprises reducing or minimizing scarring, preventing or reducing infection, inhibiting or reducing internal bleeding, inhibiting or reducing external discharge, provides protection from sun and or UV rays or a combination thereof.

3. The method of claim 1, wherein the applying the dressing is within 1 hour of the injury.

4. The method of claim 1, wherein the applying the dressing is within 2 hours, 4 hours, 8 hours, 16 hours, or 24 hours of injury.

5. The method of claim 1, wherein the wound is an incision or an open wound.

6. The method of claim 1, wherein the wound is an incision followed by suturing after surgery.

7. (canceled)

8. The method of claim 1, wherein the applying the dressing is for about 4 weeks to about 24 weeks.

9. (canceled)

10. The of claim 1, wherein the applying the dressing to the wound prevents tissue movement and decreases tension.

11. The method of claim 1, wherein the dressing allows moisture vapor permeation.

12. The method of claim 1, wherein the dressing allows oxygen permeation.

13. The method, wherein the dressing contains sun and/or UV blocking components.

14. The method of claim 1, wherein the applying the dressing results in the elimination or reduction of a risk or occurrence of a healthcare acquired infection.

15. The method of claim 1, wherein the dressing comprises a backing film and a layer coating the backing film, wherein the backing film comprises polyethylene woven fibers.

16. The method of claim 1, wherein the layer coating the backing film comprises a non-skin irritating adhesive.

17. The method of claim 1, wherein the adhesive comprises a pressure sensitive, medical grade adhesive.

18. The method of claim 1, wherein the adhesive comprises a non-woven acrylic adhesive.

19. The method of claim 1, wherein the adhesive comprises a vinyl-acrylic polymer emulsion.

20. The method of claim 1, wherein the dressing comprises antioxidants, ceramides, growth factors, vitamins, enzymes, antibiotics, antifungals, and a combination thereof.

21. The method of claim 1, wherein the dressing comprises an adhesive composition which includes a layer of pressure-sensitive adhesive composition comprising about 0.25 weight percent (wt. %) to about 15 wt. % of a high molecular weight rubber triblock copolymer, about 35 wt. % to about 65 wt. % of a superabsorbent material, 0 wt. % to about 15 wt. % of a high molecular weight diblock rubber, 0 wt. % to about 20 wt. % of an end block resin, 0 wt. % to about 40 wt. % of a diluent, 0 wt. % to about 35 wt. % of a solid tackifier, 0 wt. % to about 20 wt. % of a hydrocolloid comprising a natural product or a modified natural product, and 0 wt. % to about 10 wt. % of a semi-solid hydrocarbon.

22. The method of claim 1, wherein the subject is human.
